(19) United States
(12) Patent Application Publication
(10) Pub. No.: US 2002/0102533 A1
(43) Pub. Date: Aug. 1, 2002

(54) Hepatitis C protease exosite for inhibit or design

(76) Inventors: Charles A. Kettner, Wilmington, DE (US); Mark S. Hixon, San Diego, CA (US)

Correspondence Address: BRISTOL-MYERS SQUIBB PHARMA COMPANY, PATENT DEPARTMENT, P.O. BOX 4000, PRINCETON, NJ, 08543-4000, US

(21) Appl. No.: 09/878,579

(22) Filed: Jun. 11, 2001

Related U.S. Application Data
(60) Provisional application No. 60/210,900, filed on Jun. 11, 2000.

(51) Int. Cl. C12Q 1/70; C12N 9/50; G01N 33/53; C07K 7/00; C07K 17/00; C07K 5/00; C07K 16/00; A61K 38/04

(52) U.S. Cl. 435/5; 435/219; 435/7.1; 530/328; 530/329

(57) ABSTRACT

This invention relates to a novel method of hepatitis C protease inhibition through interaction with a novel exosite remote from the active site but overlapping with P4′-P6′ region of the extended substrate binding site. In particular, the present invention provides a description of a region of the enzyme and structure activity relationships of peptides with affinity for this exosite. Ligands binding in the exosite are competitive with larger substrates such as the physiological substrate. As such, exploitation of the exosite represents a therapeutic for the hepatitis C disease.

### FIELD OF THE INVENTION

This invention relates to a novel method of hepatitis C protease inhibition through interaction with a novel exosite remote from the active site but overlapping with P4′-P6′ region of the extended substrate binding site. In particular, the present invention provides a description of a region of the enzyme and structure activity relationships of peptides with affinity for this exosite. Ligands binding in the exosite are competitive with larger substrates such as the physiological substrate. As such, exploitation of the exosite represents a therapeutic lead for design of inhibitors of hepatitis C protease.

### BACKGROUND OF THE INVENTION

Hepatitis C, a potentially fatal liver disease, results from infection by a 9.5 kb single-stranded positive sense RNA flavivirus. At present, approximately 2% of the human population is infected with the virus. No HCV vaccine exists and the only therapy is α-interferon alone or in combination with ribavirin. Efficacy is less than 50%. Given this stark reality a major effort is underway within the pharmaceutical industry toward the discovery of an effective therapy.

Hepatitis C viral replication is initiated by the translation of a polyprotein of approximately 3,000 amino acids. Other members of the flavivirus family are yellow fever virus (YF), and animal pestiviruses like bovine viral diarrhea virus (BVDV) and swine fever virus (CSFV). Considerable heterogeneity is found within the nucleotide and encoded amino acid sequence throughout the HCV genome. At least 6 major genotypes have been characterized, and more than 50 subtypes have been described. The major genotypes of HCV differ in their distribution worldwide. The clinical significance of the genetic heterogeneity of HCV remains elusive despite numerous studies of the possible effect of genotypes on pathogenesis and therapy. All members of the Flaviviridae family have enveloped virions that contain a positive stranded RNA genome encoding all known virus-specific proteins via translation of a single, long uninterrupted, open reading frame.

These polyproteins are processed by a combination of host and viral proteolytic enzymes. For hepatitis C, nine polyproteins (C-E1-E2-NS2-NS3-NS4A-NS4B-NS5A-NS5B) are formed in the mature viron. Host proteases are responsible for the cleavage of the viral structural proteins C, E1, and E2. On the other hand, processing of mature nonstructural proteins is dependent on two viral proteases. An as yet poorly characterized Zn²⁺ dependent protease resides within the NS2 domain. It is responsible for cleavage of the NS2-NS3 juction while a serine protease, within the NS3 domain, cleaves the NS3/NS4A, NS4A/NS4B, NS4B/NS5A, and NS5A/NS5B junctions.

The NS3 protein is a 70 Kd polypeptide containing an N-terminal 21 Kd serine protease and a C-terminal 50 Kd ATP-dependent RNA helicase. The two enzymes have been cloned, expressed and characterized independently of each other. The former is described in U.S. Pat. No. 5,712,145 which discloses a recombinant purified proteolytic hepatitis C virus polypeptide comprising a defined sequence of 199 amino acids and a composition of a purified proteolytic HCV polypeptide comprising a defined sequence of 299 amino acids. De Francesco et al. in WO9522985 discloses a method for reproducing in vitro the serine protease activity associated with the HCV NS3 protein comprising the use of both sequences contained in NS3 and sequences contained in NS4A. Synthetic peptide sequences known to interact with the NS3 protease in place of full NS4a are: KKKGSVVIVGRIILSGR—NH2 (Bianchi et al. Biochemistry 36, 7890-7897, 1997) and KKGSVVIVGRIVLSGK—OH (Landro et al. Biochemistry 36, 9340-9348, 1997). Kim et al. in WO9811134 disclose crystals of hepatitis C virus protease in complex with its viral cofactor peptide. The structure of the NS3 protease region complexed to NS4a peptide has been published (Kim et al. Cell 87, 343-355, 1996). Recently, the crystal structures of competitive inhibitors bound in the P₄-P₁ sites were reported. A representative inhibitor is Boc-Glu-Leu—NH—CH(CH₂—CHF₂)C(O)—COOH. The —CH₂—CHF₂ side chain occupies the P₁ binding site and the active site serine required for bond hydrolysis complexes the ketone on the adjacent carbon (Di Marco et al. J. Biol. Chem. 275, 7152-7157, 2000). Note that the nomenclature of Schechter and Berger (Biochem. Biophys. Res. Commun. 27, 157-162, 1967) is used where P represents individual amino acids of a peptide substrate (P₆P₅P₄P₃P₂P₁-P₁′P₂′P₃′P₄′P₅′P₆′P₇′etc.) for a proteolytic enzyme which hydrolyses the peptide bond between P₁and P₁′.

More important for the present invention is the interaction of hexapeptides with HCV protease. Steinkuhler et al. (Biochemistry 37, 8899-8905, 1998) have described the binding of hydrolysis products of a 13-residue, HCV peptide substrate to the enzyme. Ac-DEMEEC—OH, Ac-EDVWAbu—C—OH, and Ac-DCSTPC—OH are reported to have Kᵢ′s of 0.6, 1.4, and 180 μM, respectively. Measurements were made using the corresponding peptides as substrates. For example, the Kᵢ of Ac-DEMEEC—OH was measured using Ac-DEMEECASHLPYK—NH₂ as a substrate in 50 mM Hepes buffer, pH 7.5, containing 1% CHAPS, 15% glycerol, 10 mM DTT and the NS4a cofactor peptide, KKKGSVWIVGRIILSGR—NH₂, at 80 μM. Binding of hexapeptides to HCV was optimized in further studies (Ingallinella et al. Biochemistry 37, 8906-8914, 1998). One of the more effective peptides was Ac-D-E-Dpa-E-Cha—C—OH for which a Ki of 0.05 μM was reported. Inhibition constants were measured by a procedure similar to those of Steinkuhler et al.(1998) except 16 pM NS4a peptide was used and Ac-DEMEECASHLPYE(Edans)—NH₂ was used as substrate. Similarly, Llinas-Brunet et al. in WO9907733 have also obtained potent inhibitors. One of their more effective compounds, Ac-Asp-(D)Glu-Chg-Val-X-Nva—OH, where X is 4-(R)-(2-naphthylmethoxy)proline, has a Ki of 0.028 μM.

The binding of Ac-D-E-Dpa-E-Cha—C—OH and related molecules to HCV protease has been studied extensively (Cicero et al. J. Molecular Biol. 289 385-396, 1999). Initially Steinkuhler (1998) and Ingallinell et al. (1998) expected hexapeptides to bind to the P₆-P₁ sites since these sites are occupied in normal substrate binding and that inhibitors were acting as competitive inhibitors of 13-mer substrate hydrolysis. NMR and modelling demonstrated that the hexapeptide binds to the protease in an extended conformation based on transfer NOE data and that it occupies the P₆-P₁ binding site.

We have discovered a novel inhibitory binding site which exists some distance from the active site of HCV protease. With a nonapeptide substrate (P6-P′3) inhibitor Q9692 occupation of this site is activating under conditions of low substrate (□Km) and low glycerol (□30%). Furthermore, occupation of this site enhances the binding of competitive inhibitors such as Q9716. The mechanism of action appears to be enhancement of substrate and inhibitor Kd. The enhancing effect is offset by increasing glycerol concentration which enhances substrate and inhibitor binding as well. In contrast, assays of enzyme activity Vs inhibitor Q9692 by use of a large (tridecapeptide) substrate do not display activation only inhibition. This would suggest that this alternate binding site overlaps with the P′4-P′6 portion of an extended binding site. Occupation of this exosite is anticipated to provide inhibition of protease activity in vivo and thus an attractive lead for inhibitor design has been discovered.

### SUMMARY OF THE INVENTION

The present invention provides a binding site of NS3 protease:NS4A complex characterized by the binding of Ac-Asp-Glu-Dpa-Glu-Cha-Cys—OH on NS3 protease in the presence of NS4A, useful for the discovery of inhibitors of HCV protease and the treatment of hepatitis C disease.

In one aspect the present invention provides for a method of evaluating a compound for utility in inhibiting hepatitis C protease. In another aspect the present invention provides for a pharmaceutical composition comprising a compound discovered using the method of evaluating a compound for utility in inhibiting hepatitis C protease. In another aspect the present invention provides for a method for treating hepatitis C comprising administering a compound discovered using the method of evaluating a compound for utility in inhibiting hepatitis C protease.

### BRIEF DESCRIPTION OF THE DRAWINGS

FIG. 1 illustrates the effect of inhibitor Q9692 on the hydrolysis of peptide substrate A (P6-P3′).

FIG. 2A illustrates the effect of inhibitor Q9692 on the hydrolysis of a P6-P3′ ester substrate.

FIG. 2B illustrates the effect of inhibitor Q9692 on the hydrolysis of a P6-P7′ substrate.

FIG. 2C illustrates the effect of inhibitor Q9692 on The hydrolysis of P6-P3′ amide substrate.

FIG. 3 illustrates the effect of NS4A peptide on the activating effect of Q9692 with a short P6-P3′ substrate.

FIG. 4A illustrates Dixon plots of 1/V versus Q9717 concentration at different fixed concentrations of Q9692 in the presence of a P6-P3′ substrate.

FIG. 4B illustrates Dixon plots of 1/V versus Q9717 concentration at different fixed concentrations of Q9692 the presence of a P6-P7′ substrate.

FIG. 4C illustrates Dixon plots of 1/V versus Q9717 concentration at different fixed concentrations of Q9714.

FIG. 5A illustrates changes in the intrinsic fluorescence of HCV Protease upon binding of NS4A peptide, Q9716(a boronic acid inhibitor)and Q9692.

### DETAILED DESCRIPTION OF THE INVENTION

The present invention provides a binding site of NS3 protease: NS4A complex characterized by the binding of Ac-Asp-Glu-Dpa-Glu-Cha-Cys—OH under physiological conditions; wherein the binding of Ac-Asp-Glu-Dpa-Glu-Cha-Cys—OH under physiological conditions is:

1) inhibitory when measured by enzymatic hydrolysis of a peptide substrate which encompasses the P6-P7′ binding sites, and

2) non-inhibitory when measured by enzymatic hydrolysis of a peptide substrate containing an ester linkage between P1-P1 which encompasses the P6-P2′ binding sites but does not extend into the P4′-P7′ binding sites region, and

3) noncompetitive inhibitory when measured by the enzymatic hydrolysis of an amide substrate encompassing the p6-p2′ region.

In a second embodiment the present invention provides for a method of evaluating a compound for utility in inhibiting hepatitis C protease comprising contacting a compound with hepatitis C protease NS3 in the presence of NS4A and a peptide substrate, wherein the peptide substrate binds to the P6-P7′ binding site, and wherein the compound binds to the binding site of Q9692, and measuring the activity of enzyme hydrolysis.

In a preferred embodiment the the hepatitis C protease NS3 is hepatitis C protease NS3 genotype 1A and the peptide substrate binds to the P2-P7′ binding site.

In a third embodiment the present invention provides for a pharmaceutical composition comprising a pharmaceutically acceptable carrier and a therapeutically effective amount of a compound identified by the method of evaluating a compound disclosed herein or a pharmaceutically acceptable salt or prodrug form thereof, wherein said compound inhibits hepatitis C protease.

In a fourth embodiment the present invention provides for a method for treating hepatitis C comprising administering to a host in need of such treatment a therapeutically effective amount of a compound identified by the method of evaluating a compound disclosed herein or a pharmaceutically acceptable salt or prodrug form thereof.

Abbreviations: As used herein: Abu means L-α-aminobutyric acid; Cha means L-cyclohexylalanine; Dpa means L-β,β-diphenylalanine; Alg means allylglycine; Nva means norvaline; boro Alg—OH means the boronic acid analog of alg where the carboxylate is replaced by —B(OH)2 (boroAlg C₁₀H₁₆) is the corresponding pmandiol ester. DMSO means dimethylsulfoxide; DTT means dithiothreitol; EDANS means 5-[2′-aminoethyl-amino]-naphthalenesulfonic acid; DABCYL is (4-(4-dimethylaminophenylazo)benzoyl; HCV means hepatitis C virus; HEPES means N-(2-hydroxyethyl)piperaxine-N′-2-ethanesulfonic acid; HPLC means high-performance pressure liquid chromatography; Maltoside means n-dodecyl-β-D-maltoside; CHAPS means 3-[3-cholamidopropyl)-dimethylammonio]-1-propanesulfonate; NS4A means (KKGSVVIVGRIVLSGKPAIIPKK); TFA means trifluoracetic acid; and Tris means tris-trihydroxymethylaminomethane.

### Materials and Methods

Peptide synthesis: The inhibitor Q9692 (Ac-D-E-Dpa-E-Cha—C—OH or Ac-Asp-Glu-Dpa-Glu-Cha-Cys—OH), its analogs, a synthetic peptide substrate Ac-D-E-M-E-E-C-A-S-H-L-P-Y-E(EDANS)—NH₂ based on the 5A-5B cleavage junction (herein defined as Peptide Substrate B), and a synthetic version of the activating peptide NS4A (KKGSVVIVGRIVLSGKPAIIPKK) were synthesized by use of solid phase Wang resin via standard Fmoc chemistry. The NS4a peptide is identical to that reported by Landro et al. (1997) except it is extended on the C-terminal by PAIIPKK. Peptide Substrate (P6-P3′ ester) A, Ac-DED(EDANS)EEAbuψ[COO]ASK(DABCYL)—NH₂ was synthesized as described by Taliani et al. (Taliani, M.; et al. Anal. Biochem., 240, 60-67, 1996). Peptide substrate C1 AC-DED(EDANS)EEAbuASK(Dabeyl)—NH2 (P6-P3′ amide) was prepared by standard procedures. The boronic acid inhibitor Q9717 (Boc-Asp (o^{t}Bu) -Glu (O^{t}Bu) -Val-Val-Pro-boroAlg—C₁₀H₆) and Q9716 (Ac-Asp-Glu-Dpa-Glu-Cha-boroAlg—C₁₀H₁₆ was prepared according to the procedure described in U.S. Provisional Patent Application No. 60/142,561, filed Jul. 7, 1999, hereby incorporated by reference.

Q9714 (Asp-Glu-Val-Val-Pro-AlgCF₂CF₃) was prepared by a procedure similar to that described by Ogilvie et.al., J. Med. Chem., 40, 4113-4135, 1997. Boc-Alg—NH(OMe)Me was prepared by a procedure known to those skilled in the art. It was reacted with CF₃CF₂⁻ Li⁺ using the procedure described by Ogilvie et. al. for the preparation of Boc-Ala—CF₂CF₃. Yield: 4.5 g, 14.2 mmol, 43.1 %. Analysis calculated for C₁₂H₁₆F₅N₃O—H: 316.3. Found: 316.1. Boc—NH—CH(allyl)—CH(OH)—CF₂CF₃ was prepared by the reduction of Boc-Alg—CF₂CF₃ with NaBH₄ also using the procedure described by Ogilvie et. al. Boc-Asp(OBu)-Glu(OBu)-Val-Val-Pro—NH—CH(Allyl)CH(OH)CF₂CF₃ was prepared by hydroxybenzatriazolyluronium coupling. Boc-Asp(OBu)-Glu(OBu)-Val-Val-Pro—OH (0.10 g, 0.13 mmol) and NH₂—CH(Allyl)CH(OH)CF₂CF₃.TFA (0.026 g , 0.12 mmol; prepared by treating the corresponding Boc compound with TFA), hydroxybenzotriazole (0.019 g, 0.045 mmol) and diisopropylethylamine (0.094 ml, 0.48 mmol) were dissolved in DMF (5 ml) and hydroxybenzatriazolyluronium (0.054 g, 0.14 mmol) were added. The reaction was allowed to stir overnight, the solvent was removed by rotoevaporation and ethyl acetate was added. The ethyl acetate solution was washed with 0.2 N HCl, 5% NaHCO₃ and brine. After drying over anhydrous MgSO₄ and concentration of organics, 0.08 g was obtained. The product was purified by silica gel chromatography using a stepwise gradient from 100% CHCl₃ to 1% MeOH in CHC1₃ to give clean product (0.048 g, 0.049 mmol, 41.9%) . Analysis calculated for C₄₄H₇₀F₅N₆O₁₂+Na: 993.5. Found: 993.5. Boc-Asp(Obu-)Glu(OBu)-Val-Val-Pro-AlgCF₂CF₃ was prepared by oxidation of the corresponding alcohol. Boc-Asp(OBu-)Glu(OBu)-Val-Val-Pro—NH—CH(Allyl)CH(OH)CF₂CF₃ (0.09 g, 0.093 mmol) was dissolved in 2 ml of 50% DMSO/toluene. Dichloroacetic acid (4.7 μl, 0.056 mmol) and dicyclohexylcarbodiimide (0.11 g, 0.56 mmol) were added and the mixture was stirred overnight. Oxalic acid (0.05 g, 0.56 mmol) was added. After 10 minutes, the reaction mixture was filtered, the filtrate was concentrated and the residue was dissolved in methanol. The product was purified by chromatography using a Sephadex column LH20 followed by HPLC using a C18 Vydac 2.2×25 cm with acetonitrile:water as gradient to give the desired product (0.06 g, 0.062 mmol, 66.6%). Analysis calculated for C₄₄H₆₉F₅N₆O₁₂ +H: 969.5. Found: 969.5. Q9714 was obtained by treating the protected peptide analog with 50% TFA in CH₂Cl₂. The final product Asp-Glu-Val-Val-Pro-AlgCF₂CF₃.TFA was purified by HPLC on a 2.2×25 cm C₁₈ Vydac column using acetonitrile:water gradient adjusted to 0.1% TFA for all solvents. Analysis calculated for C₃₁H₄₆F₅N₆O₁₀ +H: 757.5. Found: 757.5.

Enzyme preparation. The plasmid cf1SODp600, containing the complete coding region of HCV NS3 protease, genotype 1a, was obtained from ATCC (database accession: DNA Seq. Acc. M62321, originally deposited by Chiron Corporation). PCR primers were designed that allow amplification of the DNA fragment encoding the NS3 protease catalytic domain (amino acids 1 to 192) as well as its two N-terminal fusions, a 5 amino acid leader sequence MGAQH (serving as a expression tag) and a 15 amino acid His tag MRGSHHHHHHMGAQH. The NS3 protease constructs were cloned in the bacterial expression vector under the control of the T7 promoter and transformed in E. coli BL 21 (DE3) cells. Expression of the NS3 protease was obtained by addition of 1 mM IPTG and cells were growing for an additional 3 h at 25° C. The NS3 protease constructs have several fold differences in expression level, but exhibit the same level of solubility and enzyme specific activity. A typical 10 L fermentation yielded approximately 200 g of wet cell paste. The cell paste was stored at −80° C. The NS3 protease was purified based on published procedures (Steinkuhler C. et al. Journal of Virology 70, 6694-6700, 1996 and Steinkuhler C. et al. Journal of Biological Chemistry 271, 6367-6373, 1996.) with some modifications. Briefly, the cells were resuspended in lysis buffer (10 ml/g) containing PBS buffer (20 mM sodium phosphate, pH 7.4, 140 mM NaCl), 50% glycerol, 10 mM DTT, 2% CHAPS and lmM PMSF. Cell lysis was performed with use of microfluidizer. After homogenizing, DNase was added to a final concentration 70 U/ml and cell lysate was incubated at 4° C. for 20 min. After centrifugation at 18,000 rpm for 30 min at 4° C. supernatant was applied on SP Sepharose column (Pharmacia), previously equilibrated at a flow rate 3 ml/min in buffer A (PBS buffer, 10% glycerol, 3 mM DTT). The column was extensively washed with buffer A and the protease was eluted by applying 25 column volumes of a linear 0.14-1.0 M NaCl gradient. NS3 containing fractions were pooled and concentrated on an Amicon stirred ultrafiltration cell using a YM-10 membrane. The enzyme was further purified on 26/60 Superdex 75 column (Pharmacia), equilibrated in buffer A. The sample was loaded at a flow rate 1 ml/min, the column was then washed with a buffer A at a flow rate 2 ml/min. Finally, the NS3 protease containing fractions were applied on Mono S 10/10 column (Pharmacia) equilibrated in 50 mM Tris.HCl buffer, pH 7.5, 10% glycerol and 1 mM DTT and operating at flow rate 2 ml/min. Enzyme was eluted by applying 20 column volumes of a linear 0.1-0.5 M NaCl gradient. Based on SDS-PAGE analysis as well as HPLC analysis and active site titration, the purity of the HCV NS3 1a protease was greater than 95%. The enzyme was stored at −70° C. and diluted just prior to use.

Endpoint HPLC based HCV protease assay: HCV protease (5 nM) was incubated in 50 mM Tris pH 7.5, 0.1% maltoside, 5 mM DTT and appropriate amounts of glycerol, NS4A peptide, and substrate. Routine assays were 100 μl in volume containing 15% or 50% glycerol, 5.0 μM NS4A peptide and 5.0 μM substrate. Reactions were run at room temperature (˜22° C.) and quenched by the addition of 4 μl of 10% TFA before 10% of the substrate was consumed. Incubation times were 20 minutes for the ester substrate (Peptide Substrate A) and 80 minutes for the P6-P7′ amide substrate (Peptide Substrate B). For the P6-P3′ amide substrate (Substrate C) the same conditions were used except 2.0 um MS4a peptide cofactor (KKKIGSVVIVGRIILSGR—NH2) and 30nM NS₃ were used. Incubation times were 90 min. Samples, 50 μl, were injected onto an Hewlett Packard 1090 Liquid Chromatograph containing a Dynamax-60A C18 column. The column was eluted with a linear gradient from 10% acetonitrile to 45% acetonitrile in water. All solvents contained 0.1% TFA. A flow rate of 1 ml/min was used. The nonapeptide and the tridecapeptide product peaks elute at 5.2 and 8.5 minutes respectively. The product peaks were detected by fluorescence excitation at 350 nm and emission at 500 nm.

Continuous fluorescence-based HCV protease assay: This asay was a modified version of method of Taliani (Taliani, M.; et al. Anal. Biochem. 240, 60-67, 1996) using Substrate A (Ac-D-E-D(EDANS)-E-E-Abuψ[COO]-A-S-K(DABCYL)—NH₂). Assays were run in a 96-well microtiter plates using a Perkin Elmer Luminescence Spectrometer LS50B exciting at 350 nm (10 nm slit) and emitting at 500 nm (10 nm slit). In a typical assay the protease (1-4 nM) was incubated with 10 μM NS4a peptide in 50 mM Tris pH 7.0, 5.0 mM DTT, 50% glycerol, and 2% CHAPS for 15 min. Catalysis was initiated by the addition of Substrate A (final concentration 5.0 μM). Assays were also run under conditions where 15% glycerol, 0.1% Maltoside were substituted for 50% glycerol and 5% CHAPS. Enzymatic activity was monitored by measuring the increase in fluorescence with time using excitation wavelength of 350 nm and an emission wavelength of 500 nm. Both slit widths were 10 nm. Inhibition constants were determined from a nonlinear least squares fit of the data to the equation Vᵢ/Vₒ=1/(1+[I]/Kiₐₚₚ). Where Vi is the velocity measured in the presence of inhibitor (inhibitors were introduced with the NS4a peptide) and Vo is the velocity of controls. The thermodynamic Ki was determined from Kiₐₚₚₐᵣₑₙₜ via the relationship for competitive kinetics: Ki=Kiₐₚₚ/(1+[S]/Km) where [S] is the substrate concentration.

Intrinsic fluorescence studies: Assays were conducted in 0.5 ml fluorescent cuvettes (0.4 ml total volume). All assays contained 50 mM Tris pH 7.0, 15% glycerol, 0.1% Maltoside, 5 mM DTT and 450 nM HCV protease to which various amounts of NS4a peptide, active site inhibitors and Q9692 were added. Fluorescence spectra were obtained by exiting at 280 nm (10 nm slit) and scanning the emission profile from 300 to 400 nm (10 nm slit) at 1 nm s⁻¹. Samples were corrected for background fluorescence of buffer which accounted for less than 10% of the intrinsic fluorescence of the enzyme.

### Kinetic Characterization of the HCV Protease Exosite

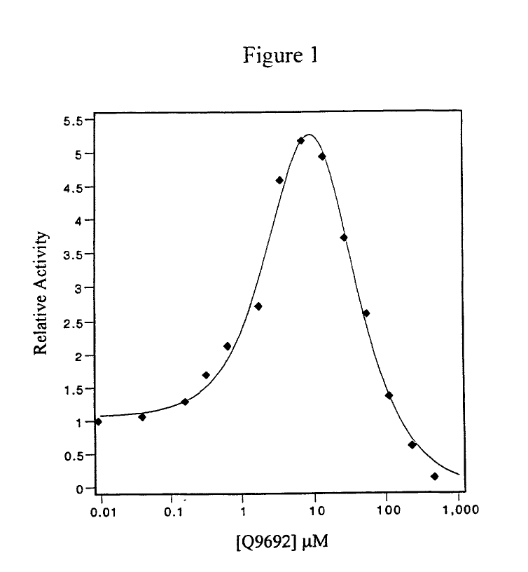

Activation: Steinkuhler et al. 1998 reported that Q9692 is a 50 nM inhibitor of HCV protease. Their studies were conducted using a 13 residue substrate spanning the region from P6-P7′. In the present invention, we have discovered that inhibition is not observed when a 9-residue ester substrate spanning the region from P6-P3′ is used. In fact ˜5-fold activation of the enzyme was observed. As shown in FIG. 1, a profile of enzyme activity with a P6-P3′ ester substrate as a function of Q9692 concentration was conducted. The presence of Q9692 between 0.2 μM to 10 μM enhanced the protease catalytic activity. Maximum stimulation (approximately 5-fold) occurs at 4 μM Q9692. At concentrations greater than 10.0 μM Q9692, inhibition was observed. An activity profile in which lower concentrations of a compound are activating while higher concentrations are inhibitory is consistent with the HCV protease possessing two distinct binding sites for Q9692. Occupation of the first site (the exosite) activates the enzyme while occupation of the second site (the P6-P1 binding site) inhibits the enzyme.

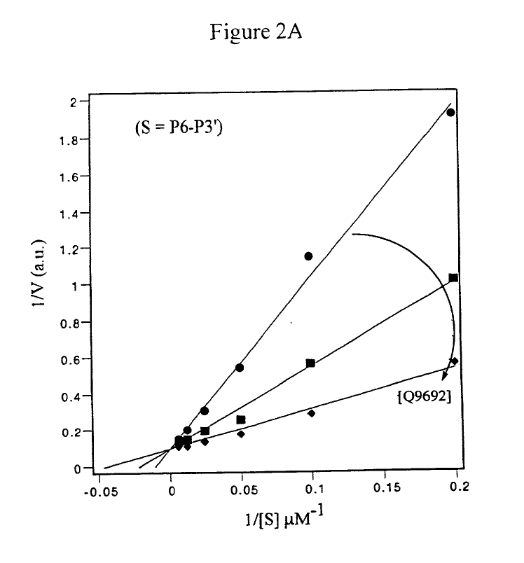

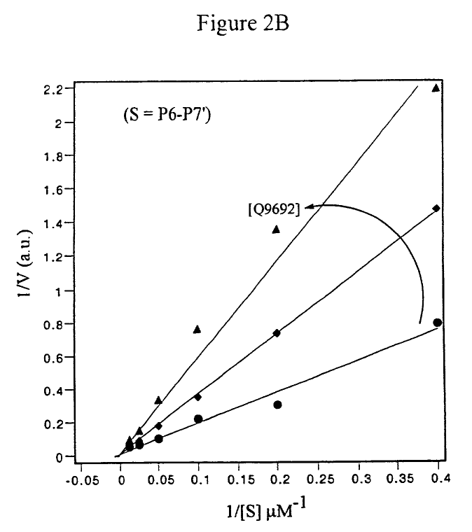

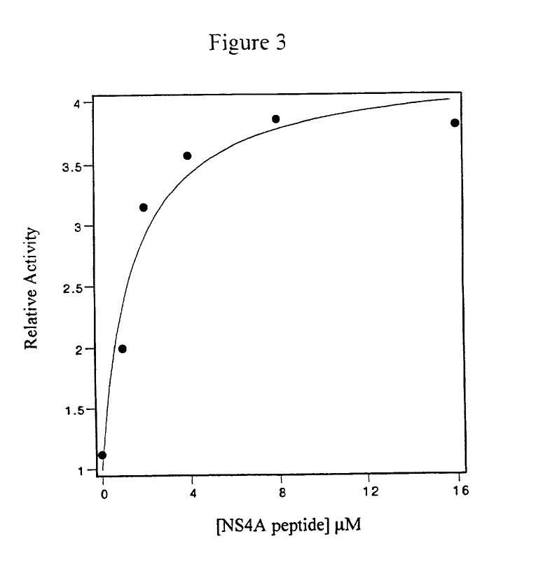

Substrate dependence: The effects of Q9692 on the enzymatic activity of HCV protease were found to be highly dependent on the length of the peptide substrate. Consistent with the results in FIG. 1, concentrations of Q9692 from 0 to 4.0 MM decrease the slope of double reciprocal plots of velocity vs substrate concentration for the P6-P3′ ester substrate (Ac-D-E-D(EDANS)-E-E-Abuψ[COO]-A-S-K(DABCYL)—NH₂), FIG. 2A. Quite different results are obtained when the larger P6-P7′ substrate (Ac-D-E-M-E-E-C-A-S-H-L-P-Y-E(EDANS)—NH₂) was used, FIG. 2B. Here Q9692 acts as a competitive inhibitor where increasing concentrations of Q9692 cause result in increases in slopes of double reciprocal plots. The Kᵢ for Q9692 obtained as a global fit was 1.9±0.3 μM. These observations (activation with a small substrate and inhibition with a large substrate) indicate a novel binding site for Q9692 (exosite) which overlaps some portion of P4′-P7′ of the normal substrate binding site.

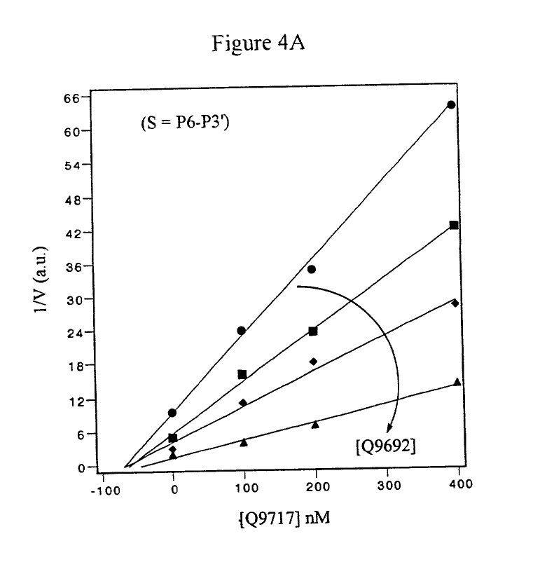

From the data in FIG. 2A, one sees that the activating effect of Q9692 is due to changes in the Km for the P6-P3′ ester substrate. For example, in the presence of 2 μM Q9692, the P6-P3′ ester substrate displays a 5-fold reduction in Kₘ (increase in substrate affinity) while k_{cat} remains relatively unchanged. Thus maximum activation is observed under conditions of low substrate concentration (S<Km) while under condition of saturating substrate, no activation by Q9692 is observed. NS4 Dependence. FIG. 3 shows the activation of protease activity on the P6-P3′ ester substrate with increasing concentration of NS4a in the presence of Q9692. Q9692 enhances the activity of the protease on this substrate, but only in the presence of NS4a (The relative activity of the protease, determined from controls run in the absence of Q9692, is ˜1 for NS4a=0).

FIG. 2C shows the effect of Q9692 on the hydrolysis of the P6-P3′ amide substrate. With increasing concentrations of Q9692 almost proportional decreases in the apparent Vₘₐₓ were observed with smaller changes in the apparent Kₘ. Here Q9692 is giving a “mixed inhibition” pattern effecting both substrate binding and catalytic efficiency. The effect of binding in the exocite is clearly differs for the two homologous ester and amide P6-P₃′ substrates where the former increase substrate binding. Regardless, a clear distinction exist between these substrates and the P6-P7′ substrate where competitive inhibition was observed.

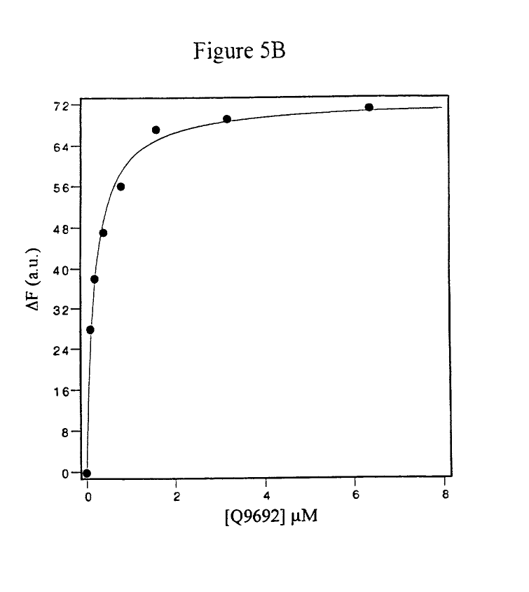

The addition of Q9692 results in a near doubling of intrinsic fluorescence. This fluorescence signal was unaltered by the addition of saturating levels of Q9716, a P6-P1 boronic acid inhibitor with a K₁ of ˜5 nM (FIG. 5A). Clearly if Q9692 and Q9716 were occupying the same site as predicted by earlier studies (Steinkuhler et al. 1998), Q9716 would displace Q9692 and reverse its effect on the fluorescence spectrum. Similarly, pre-incubation of the protease with NS4a and Q9716 does not prevent the binding of Q9692 to the protease and the accompanying increase in fluorescence (FIG. 5B).

In related experiments, the addition of Q9692 to the protease in the absence of NS4a had little effect on the fluorescence spectrum. This result is consistent with the kinetic results in FIG. 3, which shows that NS4a is required for the occupation of the exocite.

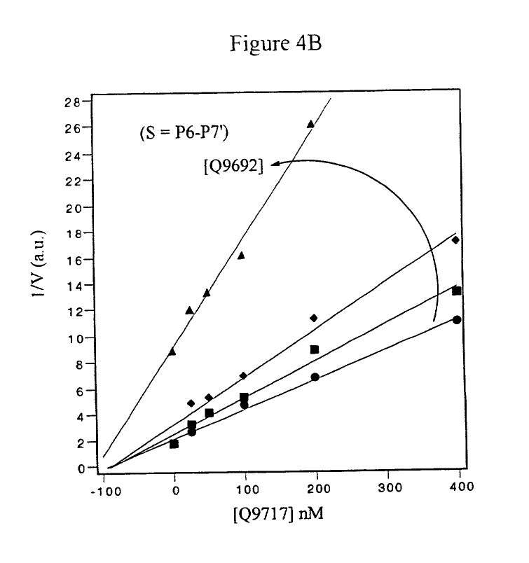

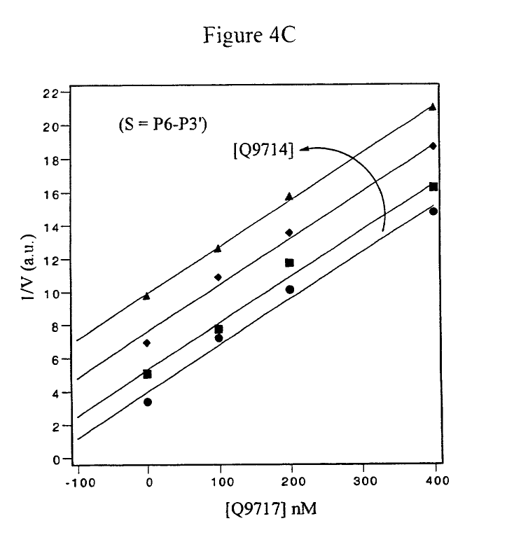

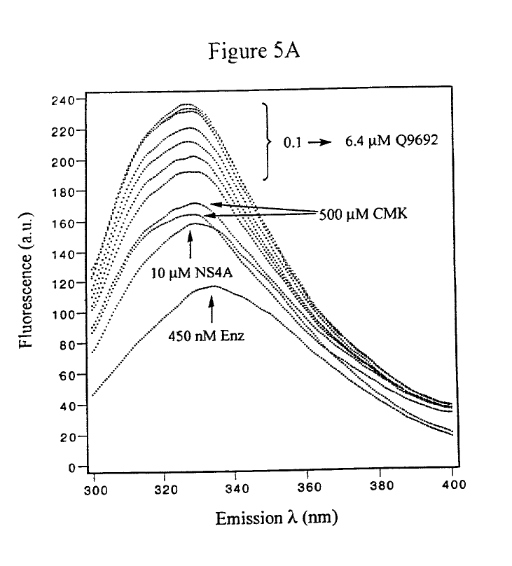

Distinguishing between the active site and the exosite. To further characterize the exosite, its behavior in the presence of a competitive inhibitor of the protease which occupies the P6-P1 binding sites (Boc-Asp(OBu)-Glu(OBu)Val-Val-Pro-boroAlg—C10H15, Q9717) was studied. As shown in FIG. 4A, a concentration matrix between Q9692 and Q9717 using the P6-P3′ ester substrate gives intersecting lines. This result indicates that Q9692 and Q9717 are binding in two independent sites (Segel, Enzyme Kinetics 1993, John Wiley & Sons, Inc., New York, N.Y., page 475). Similarly when the larger substrate (P6-P7′) is used intersecting lines are also obtained as expected, FIG. 4B. However, the concentration dependence differs from FIG. 4A, since Q9692 is activating for the smaller substrate and is inhibitory for the larger substrate. As a control for these experiments, the behavior of two compounds that possess overlapping binding sites was determined (FIG. 4C). Here Q9714 (Asp-Glu-Val-Val-Pro-AlgCF₂CF₃) was used in place of Q9692. As shown in FIG. 4C, a series of parallel lines were obtained, diagnostic for mutually exclusive binding site (binding to a single or overlapping site). Analogous binding of boronic acid inhibitors and trifluoromethyl and pentafluoroethyl ketones of similar sequence such as Q9717 and Q9714 and Q9716 are known in the art. These compounds are expected to bind in the P6-P1 sites of the enzyme. Q9717 and Q9714 were chosen arbitrarily to be used as diagnostic tools for occupation of the exosite by Q9692. Other inhibitors of HCV protease which bind in the P₆-P₁ sites and where the scissile bond is replaced by an electrophilic group can be used. These compounds are known to those skilled in the art. See Edward and Bernstein Medicinal Research Review 13, 127-194, 1994 and Mehdi Bioorganic Chemistry 21, 249-259, 1993 for examples.

SAR: A series of truncated Q9692 analogs were prepared and examined in order to determine the pharmacophore for exosite binding and inhibition of larger peptide substrates. Table 1. Briefly, the SAR is as follows: Removal of the C-terminal cysteine diminished binding some 20-fold but yielded a compound with similar activation/inhibition properties. Removal of the N-terminal Ac-Asp-Glu produced a compound with a 1 μM Kd, but the compound was activating both toward small and large substrates. The core structure Dpa-Glu-Cha appears necessary for exosite binding but this is non-overlapping with the substrate P4′-P6′ region. From the data available, the N-terminal aspartate of Q9692 appears to be the sole residue that overlaps with P4′-P6′.

Alanine scanning of Q9692 was performed. From the N-terminus, alanine substitutions produced the following: Asp→Ala reduced binding 40 fold, Glu→Ala left binding unaltered, Dpa→Ala eliminated binding, Glu→Ala left binding unaltered, Cha→Ala eliminated binding and Cys→Ala diminished binding 130 fold. The observed Cys→Ala impact on binding is 5-fold greater then the elimination of Cys in the truncation experiments. With this exception, the truncation experiments and alanine scanning experiments yielded consistent results.

TABLE 1

| | | (S = P6-P3′) | (S = P6-P3′) | (S = P6-P7′) |
|---|---|---|---|---|
| | | Dissociation | Inhibition(+)^{b} | Inhibition (+)^{b} |
| Cpd # | Structure | Constant^{a} (μM) | \overline{Activation (−)} | \overline{Activation (−)} |
| Q9692 | Ac-Asp-Glu-Dpa-Glu- | 1.0 | − | + |
| | Cha-Cys-OH | | | |
| Q228 | Ac-Asp-Glu-Dpa-Glu- | 25 | − | + |
| | Cha-NH₂ | | | |
| Q229 | Ac-Asp-Glu-Dpa-Glu- | 240 | + | + |
| | NH₂ | | | |
| Q230 | Ac-Asp-Glu-Dpa- | 400 | + | + |
| | NH₂ | | | |
| Q297 | Ac-Glu-Dpa-Glu-Cha- | 40 | − | + |
| | Cys-OH | | | |
| Q296 | Ac-Dpa-Glu-Cha-Cys- | 1.5 | − | − |
| | OH | | | |
| Q295 | Ac-Glu-Cha-Cys-OH | 300 | + | + |
| Q236 | Ac-Dpa-Glu-Cha-NH₂ | 66 | − | − |
| Q240 | Ac-Glu-Dpa-Glu-Cha- | 80 | − | − |
| | NH₂ | | | |

Footnote a for Table 1 references the assay condiditions: 1) Substrate A=P6-P3′, HCV protease (4.0 nM) was incubated with various concentrations of truncated analogs of Q9692 in 50 mM Tris buffer, pH 7.0, 15% glycerol, 0.1% maltoside, 5 mM DTT and 10 μM NS4A peptide in a 96-well microtiter plate at room temperature. After 15 minutes catalysis was initiated by the addition of Ac-D-E-D(EDANS)-E-E-Abuψ[COO]-A-S-K(DABCYL)—NH2 (final concentration 5.0 μM in a total reaction volume of 200 μl). Enzymatic activity was monitored by measuring the increase in fluorescence with time on a Perkin Elmer LS50B luminescence spectrometer (excitation 350 nm, emission 500 nm, both slits 10 nm). 2) Substrate B=P6-P7′, HCV protease (5 nM) was incubated under conditions described above with the exception that 5 μM of the substrate Ac-D-E-M-E-E-C-A-S-H-L-P-Y-E(EDANS)—NH₂ was used. Assays were 100 μl in volume, reactions were run at room temperature (˜22 C) for 80 minutes (<10% of the substrate was consumed). The addition of 4 μl of 10% TFA quenched the reactions. Quantitation of hydrolysis products was determined by measuring fluorescent peak areas following HPLC. Products were detected by excitation at 350 nm and measuring emission at 500 nm. Aliquots (50 μl) of quenched enzymatic reactions were injected on an Hewlett Packard 1090 HPLC equipped with a ¼ inch Dynamex 60A C18 column. A linear gradient from 10% acetonitrile:H2O to 45% acetonitrile:H2O was run over a period of 15 min at a flow rate of 1.0 ml/min. All solvents contained 0.1% TFA. Fluorescence products eluted at 8.5 min.

Footnote b for Table 1 references the Apparent dissociation constants: When inhibitory, dissociation constants were determined from a nonlinear least squares fit of the data to the equation V_{[I]}/Vₒ=1/(1+[I]/Kdₐₚₚ) where V_{[I]} and Vₒ are the observed velocities in the presence of specified truncated peptide concentrations and in the absence respectively, [I] is the specified concentration of inhibitor corresponding to V_{[I]} and Kdₐₚₚ is the apparent Kd under the above reaction conditions. When an analog was activating the apparent Kd was determined by fitting the saturation profile of relative velocity versus analog concentration to the equation: V_{[1]}/V_{[o]}=(Vm−1)[I]/(Kdₐₚₚ+[I])+1, where all terms have the definitions stated above and Vm is the enzyme velocity under saturating truncated peptide.

Physical Studies. In order to confirm the kinetic study presented above it was desirable to demonstrate the existence of the exosite via an independent method. The HCV protease catalytic domain undergoes large changes in its intrinsic fluorescence upon binding of NS4A, inhibitors and occupation of the exosite. As shown in FIG. 5, the binding of NS4a to the catalytic domain of NS3 produces a 25% increase in the intrinsic fluorescence when exciting at 280 nm and scanning from 300 to 400 nm. On the other hand, occupation of the exosite by Q9692 nearly doubles the intrinsic fluorescence of the enzyme alone. As with the kinetic studies, the change in intrinsic fluorescence by Q9692 was completely dependent on the presence of NS4a (FIG. 3).

### Modeling Studies

Kinetic and physical studies have shown that there is a clear distinction between Q9692 and compounds which occupy the P6-P1, P6-P3′ sites and an extended peptide which occupies the P6-P7′ site. The Q9692 binding site or exocite overlaps the P4′-P7′ substrate binding sites. To locate this site computer modeling was used. Peptide Cys-Ala-Ser-His-Leu-Pro-Tyr-Glu, which was based on the sequence of the substrate from P1 through P7′, was docked onto the surface of the HCV catalytic domain.

The solvent-accessible surface of the NS3 catalytic domain is white except for residues of the catalytic triad which are colored green (carbon), red (oxygen) and blue (nitrogen). NS4A cofactor is beige.

Protein residues having any atoms within 5 Angstroms of the modeled substrate's P3′ through P7′ are Ser5, Gln6, Gln7, Arg9, Gly10, Leu11, Cys14, Val33, Ser35, Ala37, Thr38, Asn39, Ser40, Arg107 and Lys134 of NS3A catalytic domain and Val-Gly of NS4A (of the tetrad IVGR, I'm not sure of the exact numbering in the complete sequence). These results are clearly consistent with kinetic and physical studies. Most notable is dependence of NS4A on formation of the exocite.

First molecular dynamics (using program AMBER) of the octapeptide was carried out in vacuuo in order to obtain suitable starting conformations for subsequent protein/ligand calculations. We were particularly interested in the conformation in the neighborhood of the proline residue, as its presence indicated a probable turn in the peptide backbone. Representative conformations were extracted from the dynamics trajectory.

Next residues Cys-Ala-Ser-His of these sample conformations were fit to the backbone of Leu45-Asp46-Leu47-Arg48 of Eglin. Eglin was used as a template since its conformation, when bound to chymotrypsin (PDB file 1ACB), matches closely the P4-P1 region of peptide inhibitors of HCV protease [Martin et al. (1998) Biochemistry 37, 11459-11468]. Relaxation with short molecular dynamics simulations gave probable conformations for P₁-P₃′, but not for the residues further on the prime side.

One hundred ninety-seven different orientations of residues Leu-Pro-Tyr-Glu, residues P₄′-P₇′, were generated by the Distance Geometry procedure in which the geometry of the protease and of residues P₁-P₃′ of the ligand were held constant. Each of these was allowed to reorient during 20 ps of molecular dynamics simulation. The results were clustered according to the final conformation of the four residues. The largest cluster, comprising 11 similar dockings, bound to the protein in a convincing manner. No other cluster contained more than three structures. These two observations suggest the peptide binds as indicated.

An exocite has been identified for the binding of a peptide to blood coagulation factor VIIa [Dennis et al. (2000) Nature 404, 465-470]. These are highlighted below in red, orange and yellow. Since there is fairly high degree of homology between serine proteases, HCV HS3A was superimposed on Factor VIIa. Two of the residues we suggested as belonging to its exosite are highlighted in cyan (enzyme) and beige (NS4A cofactor). This suggests that the exocites of the two enzymes are in similar locations, as are their catalytic sites.

### EXAMPLE 1

The effects of Q9692 on the hydrolysis of substrate A (P6-P3′) were determined (FIG. 1). Relative activities were determined from control runs in the absence of Q9692. HCV protease (4.0 nM) was incubated with said dilutions of Q9692 in 50 mM Tris buffer, pH 7.0, 15% glycerol, 0.1% maltoside, 5 mM DTT and 10 μM NS4A peptide in a 96-well microtiter plate at room temperature. After 15 minutes, catalysis was initiated by the addition of Ac-D-E-D(EDANS)-E-E-Abuψ[COO]-A-S-K(DABCYL)—NH2 (final concentration 5.0 μM in a total reaction volume of 200 μl). Enzymatic activity was monitored by measuring the increase in fluorescence with time on a Perkin Elmer LS50B luminescence3 spectrometer (excitation 350 nm, emission 500 nm, both slits 10 nm).

### EXAMPLE 2

Comparison of the effects of Q9692 on the hydrolysis of a P6-P3′ ester substrate (FIG. 2A) and a P6-P7′ substrate (FIG. 2B) and P6-P3′ amide substrate (FIG. 2C) were determined. For substrates A and B1 Concentrations of Q9692 are 0 (circles), 1 μM (squares), 2 μM (diamonds) and 4 μM (triangles). Assay conditions: HCV protease (5 nM) was incubated in 50 mM Tris buffer at pH 7.0, 15% glycerol, 0.1% maltoside, 5 mM DTT, 10 μM NS4A peptide, and specified concentration of substrate. Assays were 100 μl in volume and reactions were run at room temperature (22 C) and quenched by the addition of 4 μl of 10% TFA before 10% of the substrate was consumed. Incubation times were 20 minutes for the ester substrate and 80 minutes for the amide substrate. Hydrolysis products were quantitated by measuring fluorescent peak areas following HPLC and detection by excitation at 350 nm and measuring emission at 500 nm. Aliquots (50 μl) of quenched enzymatic reactions were injected on an Hewlett Packard 1090 HPLC equipped with a ¼ inch Dynamex 60A C18 column. A linear gradient from 10% acetonitrile:H2O to 45% acetonitrile:H2O was run over a period of 15 min at a flow rate of 1.0 ml/min., All solvents contained 0.1% TFA. Fluorescence products eluted at 5.2 and 8.5 min, respectively, for substrates A and B. For substrate C (FIG. 2C), similar conditions were used except rates of hydrolysis were determined in 50 mM Hepes buffer, pH 7.0, containing 2.0 μM NS4a (KKKIGSVVIVGRIILSGR—NH₂) and using 30 nM NS3 protease. Levels of substrate hydrolysis were determined after incubation for 90 min.

### EXAMPLE 3

The effect of NS4A peptide on the activating effect of Q9692 with a small substrate (S=P6-P3′) were determined (see FIG. 3). Q9692 was present in all reactions at a level of 2 μM. Enzymic activity was determined by the procedure described in FIG. 2 except levels of NS4A peptide were varied. Relative activity was calculated from control runs in the absence of NS4A peptide and Q9692.

### EXAMPLE 4

Dixon plots of 1/V versus Q9717 concentration at different fixed concentrations of Q9692 (FIG. 4A and FIG. 4B) or Q9714 (FIG. 4C) were determined. FIGS. 4A and 4C are at 5 μM substrate spanning P6-P3′ while FIG. 4B is at 5 μM substrate spanning P6-P7′. Independent binding sites are observed between Q9717 and Q9692 (intersecting lines with both substrates, FIG. 4A and FIG. 4B). Q9692 was present at 0 (circles), 1.0 μM (squares), 2 μM (diamonds), and 4 μM (triangles). Note that Q9692 is activating in FIG. 4A while inhibitory in FIG. 4B. FIG. 4C shows a model reaction between two P1-P6 competitive inhibitors (Q9717 and Q9714). The resulting parallel lines indicate that Q9717 and Q9714 are binding to the same site. Concentrations of Q9714 were 0 (circles), 12.5 μM (squares), 25 μM (diamonds) and 50 μM (triangles). Assay conditions for FIGS. 4A and 4C were as described in FIG. 1. Assay conditions for FIG. 4B were as described in FIG. 2B.

### EXAMPLE 5

Changes in the Intrinsic Fluorescence of HCV Protease on Binding of NS4a, Q9692, and a P₆-P₁ Inhibitor. Fluorescence spectrum of HCV protease (0.45 μM) in 50 mM Tris buffer, pH 7.0, containing 15% glycerol, and 0.10% maltoside were measured over a range of 300-400 nm using an excitation wavelength of 280 nm. Both excitation and emission slit widths were 10 nm and the scan rate was 1.0 nm sec⁻¹. As shown in FIG. 5A, the addition of 12.5 μM NS4a to the sample increases the intrinsic fluorescence of the protease (NS3) by 25% at 330 nm. The addition of 5.0 μM Q9692 results in a 70% further increase. Finally, the addition of 1.0 μM Q9716 (Ac-Asp-Glu-Dpa-Glu-Cha-boroAlg—C₁₀H₁₆) does not effect the spectrum. Q9716 is a potent inhibitor of HCV protease which binds in the P₆-P₁ sites. Its level in this experiment is ˜200 times its Kᵢ (˜5.0 nM)

One can conclude from these experiments that binding of Q9692 to the NS3:NS4a complex induces a large change in fluorescence. Since the spectrum is not altered by the addition of saturating levels of the boronic acid inhibitor, it is clear that Q9692 and Q9617 bind to two different independent sites.

Similar conclusions can be drawn from the data in FIG. 5B. Here the order of addition of reagents is changed. The same level of reagents are used expect the final concentration of Q9692 is 10 μM. The addition the boronic acid inhibitor to NS3 results in a small decrease in fluorescence. NS4a causes an increase in fluorescence similar to the experiment in FIG. 5A and finally the addition of Q9692 causes a large increase in fluorescence. In this case, blocking of the P₁-P₆ does not interfere with binding of Q9692 consistent with the results in FIG. 5A. Example 6.

### EXAMPLE 6

It is understood that other forms of HCV protease may be used in an assay for the determination of inhibitors. The present invention has disclosed the 1A form of HCV protease, however, the catalytic domains of types 1b, 1J and 2a have also been examined, as well as, the full-length version of form 1b. Evidence for the exosite exists in each case. Activation by Q9692 follows the trend of activation by NS4a. Forms of the enzyme that are more sensitive to NS4a display greater stimulation by Q9692 than forms that are less sensitive. In rank order sensitivity is 1J, 1B, 1A, and 2A. The catalytic activity of 2A toward the 9-mer substrate is enhanced by only 1.5-fold at 4 μM Q9692 and becomes inhibited by Q9692 at concentrations greater than 20 μM. While binding to the exosite is relatively kineticly silent versus the 9-mer substrate, against the 13-mer substrate Q9692 possesses a Ki of approximately 0.25 μM. Use of alternative forms of the HCV protease is relevent to development of an inexpensive and easy continous assay.

The presence of Q9692 in quantities sufficient to activate the enzyme also enhances the binding of several classes of competitive inhibitors (pentafluoroethyl ketones and boronic acids) by reducing their Ki's. The present invention shows that binding of Q9692 to the enzyme increases the avidity of the protease for its substrate and for substrate-like inhibitors. In contrast to the enhanced binding of inhibitors in the presence of small substrate peptides, assays with larger peptide substrates reveal only inhibition in the presence of Q9692. Exosite overlap with the P4′-P6′ provides a plausable explanation for this behavior.

### DOSAGE AND FORMULATION

The compounds determined from the present invention can be administered using any pharmaceutically acceptable dosage form known in the art for such administration. The active ingredient can be supplied in solid dosage forms such as dry powders, granules, tablets or capsules, or in liquid dosage forms, such as syrups or aqueous suspensions. The active ingredient can be administered alone, but is generally administered with a pharmaceutical carrier. A valuable treatise with respect to pharmaceutical dosage forms is Remington's Pharmaceutical Sciences, Mack Publishing.

The compounds determined from the present invention can be administered in such oral dosage forms as tablets, capsules (each of which includes sustained release or timed release formulations), pills, powders, granules, elixirs, tinctures, suspensions, syrups, and emulsions. Likewise, they may also be administered in intravenous (bolus or infusion), intraperitoneal, subcutaneous, or intramuscular form, all using dosage forms well known to those of ordinary skill in the pharmaceutical arts. An effective but non-toxic amount of the compound desired can be employed to prevent or treat neurological disorders related to modulation of a potassium channel, more specifically the M-current, formed by expression of KCNQ2 and KCNQ3 genes, such as epilepsy, anxiety, insomnia, or Alzheimer's disease.

The compounds of this invention can be administered by any means that produces contact of the active agent with the agent's site of action in the body of a host, such as a human or a mammal. They can be administered by any conventional means available for use in conjunction with pharmaceuticals, either as individual therapeutic agents or in a combination of therapeutic agents. They can be administered alone, but generally administered with a pharmaceutical carrier selected on the basis of the chosen route of administration and standard pharmaceutical practice.

The dosage regimen for the compounds determined from the present invention will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. An ordinarily skilled physician or veterinarian can readily determine and prescribe the effective amount of the drug required to prevent, counter, or arrest the progress of the condition.

Advantageously, compounds determined from the present invention may be administered in a single daily dose, or the total daily dosage may be administered in divided doses of two, three, or four times daily.

The compounds identified using the present invention can be administered in intranasal form via topical use of suitable intranasal vehicles, or via transdermal routes, using those forms of transdermal skin patches wall known to those of ordinary skill in that art. To be administered in the form of a transdermal delivery system, the dosage administration will, of course, be continuous rather than intermittant throughout the dosage regimen.

In the methods of the present invention, the compounds herein described in detail can form the active ingredient, and are typically administered in admixture with suitable pharmaceutical diluents, excipients, or carriers (collectively referred to herein as carrier materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic, pharmaceutically acceptable, inert carrier such as lactose, starch, sucrose, glucose, methyl callulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water, and the like. Moreover, when desired or necessary, suitable binders, lubricants, disintegrating agents, and coloring agents can also be incorporated into the mixture. Suitable binders include starch, gelatin, natural sugars such as glucose or β-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds determined from the present invention can also be administered in the form of liposome delivery systems, such as small unilamellar vesicles, large unilamallar vesicles, and multilamellar vesicles. Liposomes can be formed from a variety of phospholipids, such as cholesterol, stearylamine, or phosphatidylcholines.

Compounds of the present invention may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds determined from the present invention may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient and powdered carriers, such as lactose, starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol.

Suitable pharmaceutical carriers are described in Remington's Pharmaceutical Sciences, Mack Publishing Company, a standard reference text in this field.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, benzenesulfonic, toluenesulfonic, methanesulfonic, ethane disulfonic, oxalic, isethionic, and the like.

The pharmaceutically acceptable salts of the present invention can be synthesized from the parent compound identified fromthe screening assay which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, the disclosure of which is hereby incorporated by reference.

What is claimed is:

1. A method of evaluating a test compound for utility in inhibiting hepatitis C protease comprising the steps of (1) contacting the test compound with hepatitis C protease NS3 in the presence of NS4A and a peptide substrate, wherein the peptide substrate binds to a P6-P7′ binding site, and wherein the test compound binds to a binding site of Q9692, (2) and measuring the activity of enzyme hydrolysis.

2. A method of claim 1 wherein the hepatitis C protease NS3 is hepatitis C protease NS3 genotype 1A and the peptide substrate binds to a P2-P7′ binding site.

3. A pharmaceutical composition comprising a therapeutically effective amount of a test compound identified by the screening assay of claim 1 or a pharmaceutically acceptable salt or prodrug form thereof and a pharmaceutically acceptable carrier wherein said test compound inhibits hepatitis C protease.

4. A method for treating hepatitis C comprising administering to a host in need of such treatment a therapeutically effective amount of a compound identified by the screening assay of claim 1 or a pharmaceutically acceptable salt or prodrug form thereof.

5. A binding site of NS3 protease:NS4A complex characterized by the binding of Ac-Asp-Glu-Dpa-Glu-Cha-Cys—OH under physiological conditions; wherein the binding of Ac-Asp-Glu-Dpa-Glu-Cha-Cys—OH under physiological conditions is:
1) inhibitory when measured by enzymatic hydrolysis of a peptide substrate which encompasses the P6-P7′ binding sites, and
2) non-inhibitory when measured by enzymatic hydrolysis of a peptide substrate which encompasses the P6-P2′ binding sites but does not extend into the P4′-P7′ binding sites region.

6. A binding site of NS3 protease catalytic domain comprising the following residues: Ser5, Gln6, Gln7, Arg9, Gly10, Leu11, Cys14, Val33, Ser35, Ala37, Thr38, Asn39, Ser40, Arg107 and Lys134 of NS3A catalytic domain and Val-Gly of NS4A (of the tetrad IVGR).

6. A binding site according to claim 1, which does not overlap the P1-P6 binding region.

7. A method of evaluating an exocite inhibitor in which competitive inhibitions is observed for the hydrolysis of Ac-D-E-M-E-E-C-A-S-H-L-P-Y-E(EDANS)—NH₂ and at comparable levels either no inhibition or noncompetitive inhibition are observed for substrates, Ac-D-E-D(EDANS)-E-E-Abu[.COO]-A-S-K(DABCYL)—NH₂ and Ac-D-E-D(EDANS)-E-E-Abu-A-S-K(DABCYL)—NH₂, respectively.

8. A method of evaluating an exocite inhibitor comprising the steps of (1) binding P₁-P₆ inhibitors such as Ac-Asp-Glu-Dpa-Glu-Cha-boroAlg—C₁₀H₁₆ to NS3 in the presence of NS4a; and, evaluating the binding in the exocite by determining any change in intrinsic fluorescence.

9. A method of evaluating exocite inhibitor comprising the steps of: (1) binding a P₁-P₆ inhibitors such as Ac-Asp-Glu-Dpa-Glu-Cha-boroAlg—C₁₀H₁₆ to bind to NS3 in the presence of NS4a and (2) measuring the degree of binding in the exocite by displacement of Q9692 or a structurally related analog.
